(19) United States
(12) Patent Application Publication
(10) Pub. No.: US 2025/0214910 A1
(43) Pub. Date: Jul. 3, 2025

(54) MANUFACTURING NITROGEN-ENHANCED FERTILIZER

(71) Applicant: CHEMICAL EVOLUTION LTD., Calgary (CA)

(72) Inventors: Corynne O'FARRELL, Calgary (CA); Shravan TANUGULA, Calgary (CA); Alejandra ENRIQUEZ, Calgary (CA); Markus WEISSENBERGER, Calgary (CA); Kyle G. WYNNYK, Calgary (CA)

(21) Appl. No.: 18/850,170

(22) PCT Filed: Mar. 23, 2023
(86) PCT No.: PCT/CA2023/050389

§ 371 Date: Sep. 24, 2024

(30) Foreign Application Priority Data
Mar. 25, 2022 (CA) 3153386

(51) Int. Cl. C05F 17/20; C05F 1/00; C05F 5/00; C05G 5/23

(52) U.S. Cl. CPC C05F 17/20; C05F 1/005; C05F 1/007; C05F 5/002; C05G 5/23

(57) ABSTRACT

A process to prepare an aqueous fertilizer with a high nitrogen content, said process comprising the steps of: —providing a solid material having a high nitrogen content; —providing a live yeast in solution adapted to enzymatically removing nitrogen-containing compounds from said solid; —exposing said solid to said yeast in an aqueous environment, thereby creating a metabolically active culture mixture; —incubating said metabolically active culture; —injecting air in to the metabolically active mixture during said incubating step so as to inhibit the production of ethanol; wherein said metabolically active mixture undergoes said incubating step for a period of time sufficient to result in a nitrogen-fed yeast mixture; —hydrolyzing (by autolysis) the resulting nitrogen-fed yeast mixture for a period of time sufficient to remove nitrogen from said yeast, where the nitrogen removed from said yeast is present in the mixture as dissolved nitrogen.

### FIELD OF THE INVENTION

The present invention is directed to a process for dissolving solid materials rich in nitrogen, more specifically, solid waste materials rich in nitrogen for use to make fertilizer.

### BACKGROUND OF THE INVENTION

Plants which are nitrogen-deficient will have a difficulty performing photosynthesis, which affects plant growth leading to smaller than normal fruits, leaves and shoots that will develop later than usual. Telltale signs of nitrogen-deficiency in plants include, but are not limited to, yellowish or pale green leaves and weak plant growth. When a plant is in this condition, the leaves are said to be chlorotic as they can't make enough chlorophyll.

Polypeptides and proteins are highly desirable for use in agricultural fertilizers due to their high content in nitrogen. Polypeptides are basically linear chains amino acids linked together by covalent (peptide) bonds. Polypeptides occur in nature and are found in such things as meat, blood and other tissues. Because they are rich in amino acids, they have a high nitrogen content. However, meat industry waste including blood (which is dried to yield blood meal) cannot readily be used as fertilizer in agricultural setting because they are in solid from which renders the nitrogen difficult to retrieve by plants. Additionally, to be able to uptake the nitrogen in proteins, the action of proteases is needed. These proteases, typically secreted by soil microbes, break down the proteins into low molecular mass amino acids and oligopeptides that the plants can uptake. In the absence of these, like in greenhouse settings, the vast majority of plants are generally unable to uptake nitrogen in the form of large proteins.

Gelatin is made from collagen which is one of the components of various tissues such as cartilage, bone, and skin. Gelatin is mostly made up of protein containing various amino acids and polypeptides and as such, it is rich in nitrogen. Soybean meal is another high protein-based supplement that is obtained as a by-product of soybean oil extraction. As a high protein material, it is thus a high nitrogen source, usually used as animal feed. Blood meal has a nitrogen content above 13% and a phosphorous of about 1.0%. Blood meal possesses many of the valuable nutrients to make it useful as a fertilizer. Blood meal is an organic fertilizer that is made from dried animal blood. It is typically used as a nitrogen source in gardens. Blood meal is a by-product from animal processing and usually comes from slaughterhouses. It is sold as a deep reddish brown granular powder and has a strong odour. In general, blood meal does not readily dissolve in water, but some products are specifically designed to dissolved in water.

Applying blood meal to fruit bearing plants will help in the overall health of the plant. The strong odor of blood meal is also used to deter certain types of animal pests from invading gardens. Although blood meal slowly breaks down in warm, moist soil, it doesn't readily dissolve when added directly to water. Doing so generally results in a grainy, gloppy mixture which is foul smelling and does not spread well.

Water-soluble blood meal products are commercially available, these are readily dissolved in water. These are generally for concentrations 1 tablespoon per gallon and can be used as leaf spray or in irrigation driplines. However, most blood meal products which are commercially available on the market are not water-soluble. Therefore, instead of adding them to water to use as a foliar spray, they must be directly added to the soil according to package directions.

The disadvantage of spreading solid blood meal and/or bone meal is that they act much like a slow-release fertilizers and as such, will only release their full nutrient load over a period of up to a few months in certain cases. While a water-soluble form of blood and/or bone meal can allow farmers to make quick adjustments to soil levels of the missing nutrients.

Like any water-soluble fertilizer, the benefit of using a liquid form of blood meal or gelatin is that plants can access the nitrogen in it immediately, provided it can be degraded into small low molecular amino acids or oligopeptides. Contrary to granular blood meal which breaks down slowly and therefore only needs to be applied every other month or so. Many other benefits are generally attributes to blood meal because of its high nitrogen content these include, but are not limited to, growing stronger roots, encouraging greener, healthier blooms. The presence of nitrogen is a critical for plant cell growth and helps in producing verdant foliage among other things. Blood meal is a good choice of organic fertilizer but it is not balanced. Bone meal is very rich in calcium and phosphorus, and as such it is complimentary to blood meal which itself is rich in nitrogen. Gelatin is a by-product of the cattle industry. Gelatin is generally obtained by processing cattle bones, hides as well as pork and pork-related waste products (skin, for example). Different chemical processes allow to extract the collagen hydrolysate from those sources. Gelatin is relatively plentiful and low cost and provides a potentially rich nitrogen source as feedstock for the preparation of fertilizers.

Insoluble forms of blood meal or bone meal are routinely used as a type of slow-release fertilizer. Similar to blood meal, bone meal, in granular form, acts as a slow-release fertilizer, because of high phosphorus content, the primary purpose of bone meal is the development of healthy plant roots. Other solid form naturally-based options include: alfalfa meal, and feather meal.

PCT patent application WO2013013247A2 describes a fertilizer derived from an organic source and a method of making are provided. The fertilizer of the present invention advantageously has a Nitrogen content greater than 4%. The method of making the fertilizer also produces potable water. It is stated that the method for making a liquid fertilizer comprises the steps of: a) obtaining an liquid organic waste filtrate; b) adding an acid; and c) performing an evaporation process on the liquid organic waste filtrate.

U.S. Pat. No. 5,077,062 discloses a low sodium, low monosodium glutamate soy hydrolysate is prepared from a soy material, as for instance, soy flour, soy meal or soy grits by hydrolyzing the soy material with a protease enzyme in water. The hydrolysis is conducted in the absence of the addition of either acid or base at a temperature of about 90° C. for 2 hours. After deactivating the enzyme and dewatering the mixture the resulting hydrolysate contains from about 45 to about 55 weight percent of enzymatic hydrolyzed soy-based protein, from about 1 to about 3 weight percent fat, from about 5 to about 9 weight percent ash, from about 2 to about 8 weight percent water, from about 32 to about 36 weight percent carbohydrate, and less than 0.1 weight percent sodium.”

U.S. Pat. No. 5,574,139A teaches a method for the production of a powdered protein product from whole blood or a blood cell fraction separated from whole blood, the protein product being highly digestible, having a low content of iron and salts, being light brown, water-soluble and having good adhesive properties. The method for the production of the powdered protein product is characterized in that an aqueous blood cell material is subjected to hydrolysis at a temperature of between 140° and 190° C. and that the treated material is separated into a low-iron, liquid phase containing soluble proteins and an iron-rich, solid phase containing insoluble proteins, the liquid phase subsequently being concentrated or dried to a low-iron protein product, if desired.

Canadian patent CA 1,150,564 discloses a total enzymatic hydrolysate from whey proteins is disclosed comprising peptidic hydrolysate with substantially no residual proteins, at least 50% of the peptides containing 2 to 5 amino acids. 70 to 90% of the nitrogen present as peptides has a number of amino acids less than 10. There is also disclosed a process for obtaining the total enzymatic hydrolysate comprising hydrolysis of whey proteins with proteolytic enzyme (e.g. pancreatin), which is continued until there is no precipitable nitrogen with 12% trichloroacetic acid Enzymatic hydrolysis may be carried out continuously.

U.S. Pat. No. 2,906,615A teaches a process of preparing a dry, granular fertilizer from a mixture of raw untreated poultry droppings and feathers. It is stated that the steps comprise drying the mixture, introducing nitrogen, phosphoric acid and potash of fertilizer value to the mixture during the drying step, pulverizing the resultant mixture to a small particle form, adding an organic syrup and additional quantities of nitrogen, phosphoric acid and potash of fertilizer value to the mixture. The syrup is stated to act as a binder to hold the particles of the mixture in separated granular relation.

Chinese patent application 103804038A discloses a method of utilizing microorganism fermentation edible fungi residues and poultry feather to produce fertilizer, is characterized in that, comprises the following steps: (1), the pre-treatment of raw material: get edible fungi residue, by the bacterium bag fragmentation of consolidation, make it loose; (2), preparation microorganism fermenting agent: Cellulolytic bacteria is inoculated in to concussion in NB substratum and cultivates 2 days, obtain Cellulolytic bacteria microbial inoculum; Fiber degradation fungi is inoculated in to concussion in PDA substratum and cultivates 5 days, obtain fiber degradation fungal inoculant; Feather degradation bacteria is inoculated in to concussion in NB substratum and cultivates 2 days, obtain feather degradation bacterium preparation; (3), process edible fungi residue and poultry feather and produce fertilizer: the ratio that is first 1:3 by the Cellulolytic bacteria microbial inoculum in step (2) and fiber degradation fungal inoculant according to volume ratio is mixed, obtain Fibrolytic bacteria mixed bacterium, again the pretreated edible fungi residues of step (1) and Fibrolytic bacteria mixed bacterium are mixed by the mass ratio of 10:1, feather degradation bacterium preparation in poultry feather and step (2) is mixed by the mass ratio of 10:1, be that 30:1 mix with the poultry feather that is mixed with feather degradation bacterium preparation according to C/N by the edible fungi residues that is mixed with Fibrolytic bacteria mixed bacterium, the fermentation of banking up, bank up 28 days, turning once, keeps the skin wet in time weekly, and fermentation is completed fast, and bacterium slag is brown or black, and the temperature head of heap body and external environment is less than 8° C., means that fermentation completes substantially, obtains fertilizer.

Chinese patent application 101289341B discloses a preparation method of a novel biological fertilizer from processing waste of feather is characterized in that may further comprise the steps: 1. the feather processing waste material 50˜80%, plant and animal residue and livestock and poultry manure 15˜25%, and beneficial organism flora 1˜3%, all the other are water; 2. stir sealed fermenting; 3. quality inspection, according to application properties and require to add N, P, K or in, trace element; 4. deodorizing; 5. dry; 6. moulding, packing. It is stated that the method for preparing a feather-processing residue novel biological organic fertilizer includes the steps of: (1) the feather-processing residue is taken as the main raw material, and beneficial organisms flora and water are added; (2) the mixed material is stirred and hermetically fermented; (3) quality is controlled; (4) deodorization is carried out; (5) drying is carried out; (6) the fertilizer is formed and packed The method is characterized in that in the hermetical circumstance, the feather dusts rich in solid proteins are fermented and decomposed in the artificially added beneficial organisms flora, and the fermentation process kills pernicious bacteria and ascarid eggs by utilizing the interaction among bacteria to prepare the novel biological organic fertilizer with a plurality of beneficial flora. The novel biological organic fertilizer thus prepared contains abundant organic matters, beneficial flora and secondary and micro-nutrients, and can, after being applied, effectively improve the structure of soils, enhance the fertilizer conserving capacity and the water retaining capacity of soils, facilitate the releasing of the indissoluble nutrients of soils, improve the feeder capability of soils, adjust (buffer) the pH value of soils and improve the adaptability of soils.

In light of the state of the art, there still exists a need to improve the yield of nitrogen from farm waste and the like so as to allow the reuse of the extracted nitrogen, phosphorus and potassium in fertilizers, including but not limited to organic fertilizers by using yeast. This is true especially when considering the fact that most such agricultural waste that is high in nitrogen are inefficiently used and could benefit the agriculture sector in a much more substantial manner.

### SUMMARY OF THE INVENTION

According to a preferred embodiment of the present invention, there is provided a process (and method) for extracting nitrogen from solid waste stemming from agricultural activities or other industrial activity so as to produce a fertilizer which is high in nitrogen content. Preferably, the high in nitrogen fertilizer is in liquid form and can be used as such for immediate uptake by plants.

According to a preferred embodiment of the present invention, the process (and method) will enable the extraction of polypeptides and proteins from a solid material having a high nitrogen content. Preferably, said solid material having a high nitrogen content can be a material such as gelatin, blood meal, or similar high in nitrogen material which are commonly viewed or considered as side-product materials generated during other activities.

According to a preferred embodiment of the present invention, the process will employ a yeast which, itself, is the waste of another activity. Preferably, the yeast is a brewer's spent yeast as is used in breweries.

According to a preferred embodiment of the present invention, the process can employ both a brewer's spent yeast in combination with a fresh yeast sample.

According to a first aspect of the present invention, there is provided a process to prepare an aqueous fertilizer with a high nitrogen content, said process comprising the steps of:
providing a solid material having a high nitrogen content;
providing a live yeast in solution adapted to enzymatically removing nitrogen-containing compounds from said solid;
exposing said solid to said yeast in an aqueous environment, thereby creating a metabolically active culture mixture;
incubating said metabolically active culture;
injecting air in to the metabolically active mixture during an incubating step so as to inhibit the production of ethanol;
wherein said metabolically active mixture undergoes said incubating step for a period of time sufficient for said yeast to metabolize said solid material having a high nitrogen content and for said yeast to propagate and store the supplied nitrogen source in their vacuoles resulting in a nitrogen-fed yeast mixture;
hydrolyzing (by autolysis) the resulting nitrogen-fed yeast mixture for a period of time sufficient to remove nitrogen from said yeast, where the nitrogen removed from said yeast is present in the mixture as dissolved nitrogen;
optionally, recovering said dissolved nitrogen within an aqueous stream; and
optionally followed by a dehydration or evaporation step, to meet pre-determined specifications.

Preferably, the solid material having a high nitrogen content is selected from the group consisting of: blood meal; gelatin; peptone; feathers and other animal by-products; pepsin; soy protein; soybean meal; hydrolysed chitin; feather meal; and warm meal.

Preferably also, the aqueous environment further contains a carbohydrate. More preferably, the carbohydrate is selected from the group consisting of: glucose; sucrose; fructose and combinations thereof.

According to a preferred embodiment of the present invention, the conditions of said aqueous environment are adapted to minimize the production of ethanol. Preferably, the carbohydrate is present in an amount ranging from 0.25 wt % to 25 wt % of the total weight of the mixture.

According to a preferred embodiment of the present invention, the live yeast is a brewer's spent yeast.

According to a preferred embodiment of the present invention, the live yeast is a brewer's spent yeast supplemented by another yeast culture (to “spike” a BSY batch that might have a low cell count).

According to yet another preferred embodiment of the present invention, the live yeast is a brewer's spent yeast supplemented by another yeast culture and additional nutrients.

According to another preferred embodiment of the present invention, the live yeast culture is a not brewer's spent yeast.

According to a preferred embodiment of the present invention, the incubating of said metabolically active culture is carried out at a temperature ranging from 20-40° C. for a period of 12-48 hours.

According to a preferred embodiment of the present invention, the incubating of said metabolically active culture is carried out at a temperature ranging from 25-35° C. for a period of 12-48 hours.

According to a preferred embodiment of the present invention, the lysis step is carried out at a temperature ranging from 40-60° C. for a period of minimum 24 hours, preferably between 24 and 48 hours.

According to a preferred embodiment of the present invention, the lysis step is carried out at a temperature ranging from 45-55° C. for a period of minimum 24 hours, preferably between 24 and 48 hours.

According to a preferred embodiment of the present invention, the nitrogen being removed from said yeast is in the form of polypeptides, proteins and/or amino acids. Preferably, the nitrogen is dissolved in the aqueous solution. More preferably, the dissolved nitrogen can readily be used as a fertilizer by applying to roots of a plant in need thereof, near the roots of a plant in need thereof. According to a preferred embodiment of the present invention, the dissolved nitrogen can readily be used as a fertilizer by a foliar spray onto the leaves of plant in need thereof.

According to a first aspect of the present invention, there is provided an aqueous composition for use as a fertilizer, wherein said composition comprises a soluble nitrogen content ranging from 1 to 15% in the form of a nitrogen source selected from the group consisting of: yeast; proteins; polypeptides; and amino acids as soluble components. Preferably, said nitrogen source is derived from a natural source selected from the group consisting of: blood meal; gelatin; peptone; feathers and other animal by-products. Preferably, the yeast is brewer's spent yeast.

More preferably, said nitrogen is derived from a combination of natural source selected from the group consisting of: yeast and blood meal; gelatin; peptone; feathers and other animal by-products.

According to a preferred embodiment of the present invention, the external nitrogen sources that are added to live yeast are converted by the yeast to small molecules by yeast vacuolar proteases Cerevisin (EC 3.4.21.48, yeast proteinase B, proteinase yscB, baker's yeast proteinase B, brewer's yeast proteinase, peptidase beta) are active in hydrolysate and can be utilized to degrade protein like gelatin to bring it into the solution.

### DETAILED DESCRIPTION OF THE INVENTION

The description that follows, and the embodiments described therein, is provided by way of illustration of an example, or examples, of particular embodiments of the principles of the present invention. These examples are provided for the purposes of explanation, and not limitation, of those principles and of the invention.

According to a preferred embodiment of the present invention, there is provided a process to prepare an aqueous fertilizer with a high nitrogen content, said process comprising the steps of:
providing a solid having a high nitrogen content;
providing a yeast adapted to enzymatically attack said solid; and
exposing said solid to said yeast in an aqueous environment, for a period of time sufficient for said yeast to break down and dissolve said nitrogen from said solid;
wherein the dissolved nitrogen is recovered from an aqueous stream.

According to a preferred embodiment of the present invention, the resulting product may be dried and applied directly to the soil around the plant as a dry fertilizer.

According to a preferred embodiment of the present invention, the resulting product may be applied by irrigation or solution.

According to a preferred embodiment of the present invention, the resulting product may be applied directly as a foliar application as a spray, powder or dust.

According to a preferred embodiment of the present invention, the resulting product may be applied at any time including the time of planting or transplanting of seeds or plants up to time of harvesting of crops such as fruits, vegetables, grasses, and the like.

According to a preferred embodiment of the present invention, fertilizers obtained can overcome a common drawback of commercially available fertilizers which require the presence of an organism such as a bacterium present on the plant to release the nitrogen contained on a nitrogen-containing ingredient into a nitrogen form usable by the plant. Some examples of nitrogen-containing ingredient include: calcium nitrate, urea formaldehyde-based compounds, nitrocellulose, ammonium nitrate, ammonium sulfate, urea and derivatives thereof, monoammonium phosphate, diammonium phosphate, just to name a few.

According to a preferred embodiment of the present invention, fertilizers are substances containing at least one of the plant nutrients nitrogen, phosphate or potassium. According to another embodiment of the present invention, the fertilizer may be mixed with additives such as: soil nutrients; natural organic products; algicides, bactericides, defoliants, fungicides, herbicides, insecticides, miticides, nematicides, pesticides, repellents, rodenticides and combinations thereof. According to another embodiment of the present invention, the fertilizer may be mixed with additives such as: biostimulants which promote the health and survival of a plant, wherein said biostimulant is selected from the group consisting: cytokinins, auxins, gibberellins, ethylene, abscisic acid and a combination of these. According to a preferred embodiment of the present invention, the natural organic product is selected from the group consisting of: humic acid; blood meal; bone meal; seed meal; feather meal; and soy meal; meat meal; animal waste from various animal sources; activated sludge, hydrolyzed animal hair; fish by-products; chitin; composts; and a combination thereof.

### Experiments Based on Brewer's Spent Yeast

According to a preferred embodiment of the present invention, BSY is obtained from a local brewery which is a waste product for most of the breweries. Yeast (mainly S. Cerevisiae strain) has four growth phases that are lag phase, exponential phase, stationary phase and death phase.

Fresh BSY when obtained from the brewery is generally in a stationary phase and contains wort, which is mostly starch, and small amounts of fermentable carbohydrate sources (contents varies based on the fermentation conditions and supply chain delay). The nitrogen content for dry BSY was measured to be in the range of 7 to 9%.

BSY is a by-product of the brewing industry. According to a preferred embodiment of the present invention, a quantity of nitrogen supplement that can be added to said BSY is selected from a group consisting of one of various solids having a high-nitrogen content. Preferably, the yeast is left for a period of approximately 12-48 hours to incubate under aerobic conditions to inhibit or substantially reduce the undesirable production of ethanol. As the person skilled in the art would understand, avoiding the production of ethanol in the present method is highly desirable as it allows for a greater production of soluble nitrogen-containing compounds such as oligopeptides, amino acids or the like which are valuable for use as fertilizer, which ethanol is not. According to a preferred embodiment of the present invention, the aerobic conditions can be achieved through a variety of ways including, but not limited to, the bubbling (or injection) of air into the vessel where the yeast, sugar (source of carbohydrate) and source of nitrogen are mixed together. Having an open top vessel for the incubation may partially achieve this goal but may not be sufficient to inhibit the production of ethanol. During that period, the yeast extracts the nitrogen in the solids having a high-nitrogen content to create further proteins, polypeptides, amino acids or the like which are sources of readily bioavailable nitrogen for plants and the like. At the end of the 12-48 hours, the yeast is hydrolyzed. According to a preferred embodiment of the present invention, after lysis, if required, the hydrolyzed yeast is administered an additional mixture of amino acids to further increase the nitrogen content in the liquid solution of hydrolyzed yeast.

According to a preferred embodiment of the present invention, the BSY is incubated in the presence of nutrients. The obtained BSY, which is in stationary phase, which is generally a 5-30% slurry, more preferably a 15-17% slurry (yeast+solids) and up to 95% liquid (water), is revitalized by the addition of some ingredients and the brew is brought back to the exponential phase, where the yeast multiplies under aerobic conditions to reduce ethanol production and increase cell growth in presence of a carbohydrate source and extra nitrogen source for next 12-48 hours. The nitrogen is consumed by the yeast partly for propagation and partly it is stored in the vacuoles of yeast cells.

This step increases the cell count and thus, contributes to the total nitrogen content. The vitamins and minerals required by the yeast are supplied from the spent liquid wort. This would eventually run the wort to dry conditions by using all available carbohydrate sources and nutrients in presences of excess nitrogen.

According to a preferred embodiment of the present invention, subsequent to the incubation of the yeast follows a step of lysis of the yeast. Preferably, after 24 hours of revitalized exponential growth phase, the yeast enters back to the stationary phase, after which the broth is heated to 45-55° C. for next 24-48 hours, where the yeast internal enzymes promote autolysis of the cells releasing proteases into the liquid. As BSY contains about 48% protein content, the proteases act upon the proteins and cleave them and generates smaller amino acids. The same proteases also dissociate any leftover proteins like gelatin, that is not completely consumed during the growth that are added to the broth. Thus, increasing the overall nitrogen content. At this stage, the broth is separated into two phases, the top layer is the water-soluble phase with dissolved proteins, amino acids, nucleic acids etc. and the bottom is the flocculated insoluble layer or cell walls and carbohydrate wort from the BSY-wort mixture.

According to a preferred embodiment of the present invention, subsequent to the lysis step the top layer is collected. This fraction when collected has 0.2 to 4% nitrogen. Preferably, up to 60-66% of water is removed, in some cases the percentage is even higher. The liquid is concentrated to obtain higher nitrogen content (up to 12%), phosphorus and potassium contents up to 1% respectively, as a natural or totally organic source of liquid fertilizer. According to a preferred embodiment of the present invention, the above-mentioned liquid fertilizer can be further formulated by the addition of excess nitrogen, phosphorus and potassium to obtain any required concentrations of NPK values.

### EXPERIMENTAL

Various experiments were carried out to determine the impact of using yeast to enhance the nitrogen content of fertilizer. The nitrogen testing is carried out using the Dumas method AOAC 993.13 and the Kjeldahl method AOAC 962.10. The results of those experiments are compiled in Table 1.

### Incubation in the Presence of Nutrients

A few different experiments were conducted where BSY was incubated with different sources of nitrogen which present themselves in a solid state.

It is known from literature that yeast vacuolar proteases such as Cerevisin (EC 3.4.21.48, yeast proteinase B, proteinase yscB, baker's yeast proteinase B, brewer's yeast proteinase, peptidase beta) are active both in the active yeast as well as in the hydrolysate and can be utilised to degrade protein like gelatin to bring it into the solution which can be seen in experiments C-106 to C-108 as well as Al in comparison to controls C-112 to C-114, C-74 and A-5.

The yeast and wort from BSY were mixed well and:
500 g of the BSY and 200 g of water were incubated for 24 hours at 30° C. with no additional nutrients and lysed after by placing it at 50° C. for 24 hours. After lysis, the mixture was filtered and evaporated to approximately 33% of the original volume.
500 g of the BSY were incubated for 24 hours at 30° C. with 200 g of water and 10 g of a carbohydrate (i.e., sucrose, glucose, fructose, etc.). After the incubation period, the yeast was lysed by placing it at 50° C. for 24 hours. After lysis, the mixture was filtered and evaporated to approximately 33% of the original volume.
700 g of water were incubated for 24 hours at 30° C. with 25 g of a carbohydrate (i.e., sucrose, glucose, fructose, etc.). After the incubation period, the mixture was placed at 50° C. for 24 hours; after which, the mixture was filtered and evaporated to approximately 33% of the original volume.

According to another embodiment of the present invention, BSY and water were incubated with 12.5 g of gelatin and a source of carbohydrate. The BSY was grown for 24 h before lysis and the hydrolysate was filtered and concentrated by evaporation of 66% water. Total nitrogen analysis was done on the concentrate and extrapolated to the original sample.

TABLE 1
Summary of experiments using gelatin and 25 g of carbohydrate.

| | | N % in |
|---|---|---|
| | | evaporated |
| Sample ID | Feed Additives | Sample |
| A-5 | 500 g BSY + 200 g water | 1.57 |
| C-74 | 700 g water + 25 g glucose + 12.5 g gelatin | 0.77 |
| A-1 | 500 g BSY + 200 g water + 25 g glucose + 12.5 | 2.91 |
| | g gelatin | |

TABLE 2
Summary of experiments using gelatin and 10 g of carbohydrate.

| | | N % in | Mass removed |
|---|---|---|---|
| Sample | | evaporated | during |
| ID | Feed Additives | Sample | filtration (g) |
| C-112 | 500 g BSY + 200 g water + 10 g | 1.24 | 207.82 |
| | sucrose | | |
| C-108 | 500 g BSY + 200 g water + 10 g | 2.45 | 215.58 |
| | sucrose + 12.5 g gelatin | | |
| C-115 | 500 g BSY + 200 g water + 10 g | 1.51 | 316.09 |
| | sucrose + 12.5 g gelatin (no | | |
| | incubation) | | |

The results are clear that, in the presence of yeast (experiment A-1), there was an increased nitrogen content compared to cases where the yeast or gelatin was not added (experiments C-74 and A-5, respectively). There was a clear indication that the presence of yeast with a solid complex nitrogen-containing compound resulted in the extraction of nitrogen therefrom and hence solubilization of previously solid and difficult to solubilize nitrogen. This occurs through the concentration of existing nitrogen by the yeast using aerobic respiration rather than fermentation. From Table 2, it is clear that the incubation step (experiment C-108) is critical for the yeast to extract and concentrate the nitrogen from the gelatin in comparison with the controls (experiments C-112 and C-115). Additionally, during the filtration step, an increase amount in mass removed was observed when filtering C-115 than C-108, which highlights the effect of the yeast in solubilizing the nitrogen from the gelatin.

Table 3 provides a summary of the results of an experiment carried out in which blood meal was used as the high nitrogen compound solid.

TABLE 3
Summary of experiments using blood meal.

| | | N % in |
|---|---|---|
| Sample | | evaporated |
| ID | Feed Additives | Sample |
| C-73 | 700 g water + 25 g glucose + | 0.01 |
| | 12.5 g blood meal | |
| C-112 | 500 g BSY + 200 g water + 10 g | 1.24 |
| | sucrose | |
| C-116 | 500 g BSY + 200 g water + 10 g | 0.13 |
| | sucrose + 15 g blood meal (no | |
| | incubation) | |
| C-109 | 500 g BSY + 200 g water + 10 g | 1.88 |
| | sucrose + 12.5 g blood meal | |

The data set out in table 3 demonstrates that blood meal being a non-water soluble solid cannot be detected by the method when in water or when applied to the yeast without the incubation step; therefore resulting in almost negligible % N testing results (experiments C-73 and C-116). This nitrogen in solid form will not uptake as readily and easily by plants, causing fertilizers to not be equally efficient as soluble forms of nitrogen.

For the experiment where yeast was present and allowed to grow in the presence of blood meal (experiment C-109), there was a clear indication that the presence of yeast with a solid complex nitrogen-containing compounds resulted in the extraction of nitrogen therefrom and hence solubilization and concentration of previously solid and difficult to solubilize nitrogen.

Table 4 provides a summary of the results of an experiment carried out in which soy meal and peptone were used as the high nitrogen compound solid.

TABLE 4
Summary of experiments using soy meal and peptone.

| | | N % in | N % in |
|---|---|---|---|
| Sample | | 100% | evaporated |
| ID | Feed Additives | Sample | Sample |
| D-1 | 500 g BSY + 200 g water + 10 g | 0.51 | 1.54 |
| | sucrose | | |
| D-8 | 500 g BSY + 200 g water + 10 g | 0.62 | 1.85 |
| | sucrose + 12.5 g peptone from | | |
| | milk solids | | |
| D-11 | 500 g BSY + 200 g water + 10 g | 0.68 | 2.05 |
| | sucrose + 12.5 g soy meal | | |

The data set out in table 4 demonstrates that the samples where the yeast was incubated with peptone and soy meal, both of which typically having limited solubility in water, yielded increased nitrogen. The data clearly indicates that the presence of yeast resulted in the extraction of nitrogen therefrom and hence solubilization and concentration of previously solid and difficult to solubilize nitrogen.

In light of the above experimental data obtained, one can obtain a liquid fertilizer having a high nitrogen content by exposing a yeast to a solid form of complex nitrogen-rich materials including but not limited to gelatin, or blood meal.

According to a preferred embodiment of the present invention, there is provided a method to reliably increase the available nitrogen in liquid form for use in fertilizing plants in need of such. Preferably, the nitrogen used in the fertilizer is extracted from nitrogen-rich solids which are generally considered as waste products from agricultural or related industries, including but not limited to, cattle, pork and other livestock.

According to a preferred embodiment of the present invention, there is provided a method to reliably increase the available nitrogen in liquid form for use in fertilizing plants in need of such wherein said nitrogen comes from feathers such as, but not limited to, chicken feathers.

It is noteworthy to point out that chicken feathers are being incorporated in solid form with other elements to create fertilizers. However, the present invention allows to extract the nitrogen contained in chicken feathers and have it in liquid form and thus readily available as opposed to the other stated method where chicken feathers are simply ground up and incorporated with other components and spread onto fields in solid form.

The approach of grinding chicken feathers and spreading them, in a solid form, onto a filed to be fertilized renders the nitrogen contained therein difficult to extract and, thus, not a viable option for immediate increase of nitrogen in the soil and/or around or in close proximity to plants and the like requiring an immediate boost of nitrogen.

Moreover, it has not been established that chicken feather waste is a good source of nitrogen over time. A process which can ensure the solubilization of the nitrogen contained in such waste would ensure that the nitrogen extracted is available as a source for plants requiring such.

According to a preferred embodiment of the present invention, there is a substantial advantage in the ability to solubilize nitrogen contained in blood meal so as to make it available for immediate uptake by plants and the like as opposed to having blood meal be spread onto fields as a ‘delayed release’ source of nitrogen. Many things can happen over the course of a growing season which would impact the solid blood meal's presence on (or in) the soil. Flash floods and the like may sweep away the blood meal and thus remove the presence of such high in nitrogen sources meant for the plants. In such situations, it would be beneficial for growers to add liquid blood meal to their crops on multiple occasions so as to ensure uptake thereof by the plants, as opposed to a single application of solid blood meal which may be washed away.

According to a preferred embodiment of the present invention, the process will enable the extraction of polypeptides and proteins from a solid material generated by the food production industry where said solid material is not prioritized by animal feed manufacturers.

According to an aspect of the present invention, there is provided a process to prepare an aqueous fertilizer with a high nitrogen content, said process consisting of the steps of:
providing a solid material having a high nitrogen content;
providing a live yeast in solution adapted to enzymatically removing nitrogen-containing compounds from said solid;
exposing said solid to said yeast in an aqueous environment, thereby creating a metabolically active culture mixture;
incubating said metabolically active culture;
injecting air in to the metabolically active mixture during an incubating step so as to inhibit the production of ethanol;
   wherein said metabolically active mixture undergoes said incubating step for a period of time sufficient for said yeast to metabolize said solid material having a high nitrogen content and for said yeast to propagate and store the supplied nitrogen source in their vacuoles resulting in a nitrogen-fed yeast mixture;
hydrolyzing (by autolysis) the resulting nitrogen-fed yeast mixture for a period of time sufficient to remove nitrogen from said yeast, where the nitrogen removed from said yeast is present in the mixture as dissolved nitrogen;
optionally, recovering said dissolved nitrogen within an aqueous stream; and
optionally followed by a dehydration or evaporation step, to meet pre-determined specifications.

According to a preferred embodiment of the present invention, the process allows for direct addition and impregnation of nitrogen and sugar sources with yeast for the production of both organic liquid and solid fertilizers. This process is robust and able to enrich the nitrogen content of fertilizers to values ranging from 1 to 12% without resorting to using inorganic nitrogen salts. Preferably, this process produces no waste and converts all feed materials into valuable environmentally-friendly liquid and/or solid fertilizers. Preferably, the process starts by obtaining the BSY waste product from a brewery to utilize the yeast and starchy wort for the incubation stage. This stage is followed by the growth and multiplication of yeast in which it multiplies under aerobic conditions to reduce ethanol production and increase cell growth in the presence of a novel drop-in technique of adding sugar and nitrogen sources (such as but not limited to gelatin, blood meal). Finally, the growth reaches the targeted stage, the whole broth including yeast is heated to 45° C. for 24 hours.

According to another aspect of the present invention, there is provided a process to prepare an aqueous fertilizer with a high nitrogen content, said process comprising the steps of:
providing a solid material having a high nitrogen content;
providing a live yeast in solution adapted to enzymatically removing nitrogen-containing compounds from said solid;
exposing said solid to said yeast in an aqueous environment, thereby creating a metabolically active culture mixture;
optionally, adding other soluble high nitrogen compounds to increase the nitrogen content.
incubating said metabolically active culture;
injecting air in to the metabolically active mixture during said incubating step so as to inhibit the production of ethanol;
   wherein said metabolically active mixture undergoes said incubating step for a period of time sufficient for said yeast to metabolize said solid material having a high nitrogen content and for said yeast to propagate and store a portion of the supplied nitrogen source in their vacuoles resulting in a nitrogen-fed yeast mixture;
optionally, adding other soluble high nitrogen compounds to increase the nitrogen content.
hydrolyzing (by autolysis) the resulting nitrogen-fed yeast mixture for a period of time sufficient to remove nitrogen from said yeast, where the nitrogen removed from said yeast is present in the mixture as dissolved nitrogen;
optionally, adding other soluble high nitrogen compounds to increase the nitrogen content.
optionally, recovering said dissolved nitrogen within an aqueous stream; and
optionally followed by a dehydration or evaporation step, to meet pre-determined specifications.

Preferably, the high nitrogen compound to increase the nitrogen content is selected from the group consisting of: Lysine (Lys); Methionine (Met); Tryptophan (Trp); Arginine (Arg); Histidine (His); Isoleucine (Ile); Leucine (Leu); Phenylalanine (Phe); Threonine (Thr); Valine (Val); Glycine (Gly); Cystine (Cys); Tyrosine (Tyr); Alanine (Ala); Glutamine (Gln); Glycine (Gly); Serine (Ser); Asparagine (Asn); Aspartic Acid (Asp); and Glutamic Acid (Glu); and a combination thereof and/or salts thereof.

According to a preferred embodiment of the present invention, the process produces a two-phase broth (Liquid/Solid) which is separated where the liquid is concentrated through evaporation and yielded the liquid fertilizer with a nitrogen content of 1-12%. The solid is sent to a rotary dryer unit and yields a solid organic fertilizer flake.

According to a preferred embodiment of the present invention, the carbohydrate added to the yeast and to the nitrogen-rich material can be added in batches, or in continuously or almost continuously fashion using a drip method or the like.

It is well understood in the field that solid materials used as fertilizers offers a much slower release of nutrients compared to a fertilizer in liquid form. Solid fertilizers have a bioavailability which is dependent on the presence of bacteria in the soil as well as temperature and rain in order to be broken down and taken up by plants. In some cases, this process of solubilizing the solid material can take up to a year depending on the type of solid.

According to a preferred embodiment of the present invention, it is possible to use waste products in the preparation of fertilizers to increase the nitrogen content of such. Preferably, a method according to a preferred embodiment of the present invention, allows for the extraction of nitrogen from solids including but not limited to, peptones and soy meal to increase the nitrogen content in fertilizers. Such solid materials may have conventionally been scattered on fields as fertilizers or soil amendments but, in such instances, their plant bioavailability is greatly reduced as they require the presence of bacteria in the soil to breakdown large proteins to release amino acids or other forms of nitrogen containing compounds (i.e., inorganic nitrogen) and make the latter bioavailable for uptake by plants.

According to a preferred embodiment of the present invention, the liquid fertilizer resulting from the process is made in part from waste material. Preferably, the liquid fertilizer does not contain any inorganic salts. Preferably also, the liquid fertilizer further contains amino acids. Preferably, said amino acids are solubilized.

According to a preferred embodiment of the present invention, the liquid fertilizer resulting from the process is useful in settings where plants are grown in environments where soil bacterial content is low or absent (such as hydroponics). Preferably, the aqueous fertilizer with a high nitrogen content overcomes the absence of bacteria by providing bioavailable forms of nitrogen to the plants.

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

1. A process for preparing an aqueous fertilizer with a high nitrogen content, said process comprising the steps of:
a) providing a solid material having a high nitrogen content;
b) providing a live yeast in a solution adapted for enzymatically removing nitrogen-containing compounds from said solid material;
c) exposing said solid material to said yeast in an aqueous environment, thereby creating a metabolically active culture mixture;
d) incubating said metabolically active culture mixture;
e) injecting air into the metabolically active culture mixture during said incubating step so as to inhibit the production of ethanol;
wherein said metabolically active culture mixture undergoes said incubating for a period of time sufficient for said yeast to metabolize said solid material and for said yeast to propagate and store a portion of the nitrogen resulting in a nitrogen-fed yeast mixture;
f) hydrolyzing the resulting nitrogen-fed yeast mixture for a period of time sufficient to remove nitrogen from said yeast, wherein the nitrogen removed from said yeast is present in the nitrogen-fed yeast mixture as dissolved nitrogen;
g) optionally, recovering said dissolved nitrogen within an aqueous stream; and
wherein the hydrolyzing is optionally followed by dehydrating or evaporating.

2. The process according to claim 1 wherein the solid material is selected from blood meal, gelatin; peptone, feathers and other animal by-products, pepsin, soy protein, hydrolysed chitin, feather meal, bone meal and warm meal, and combinations thereof.

3. The process according to claim 1, wherein the aqueous environment further contains a carbohydrate.

4. The process according to claim 3, wherein the carbohydrate is selected from glucose, sucrose, fructose, and combinations thereof.

5. The process according to claim 1, wherein the conditions of said aqueous environment are adapted to minimize the production of ethanol.

6. The process according to claim 3, wherein the carbohydrate is present in an amount ranging from 0.25 wt % to 25 wt % of the total weight of the metabolically active culture mixture.

7. The process according to claim 1, wherein the live yeast is a brewer's spent yeast.

8. The process according to claim 7, wherein the live yeast is a brewer's spent yeast that is supplemented by another yeast culture.

9. The process according to claim 7, wherein the live yeast is a brewer's spent yeast that is supplemented by another yeast culture and additional nutrients.

10. The process according to claim 1, wherein the live yeast culture is not a brewer's spent yeast.

11. The process according to claim 1, wherein the incubating of said metabolically active culture mixture is carried out at a temperature of 20-40° C. for a period of 12-48 hours.

12. The process according to claim 1, wherein the incubating of said metabolically active culture mixture is carried out at a temperature of 25-35° C. for a period of 12-48 hours.

13. The process according to claim 1, wherein the hydrolyzing step is carried out at a temperature of 40-60° C. for a minimum period of 24 hours.

14. The process according to claim 1, wherein the hydrolyzing step is carried out at a temperature of 45-55° C. for a minimum period of 24 hours.

15. The process according to claim 1, wherein the nitrogen removed from said yeast is in the form of polypeptides and proteins.

16. An aqueous fertilizer composition comprising a soluble nitrogen content in the form of a nitrogen source selected from yeast, proteins, polypeptides, and amino acids; wherein the soluble nitrogen content is from 1 to 15% of the composition.

17. The aqueous fertilizer composition according to claim 16, wherein said nitrogen source is derived from a natural source selected from blood meal, gelatin, peptone, feathers, other animal by-products, and combinations thereof.

18. (canceled)

19. The aqueous fertilizer composition according to claim 17, wherein the natural source is yeast.

20. The aqueous fertilizer composition according to claim 19, wherein the yeast is brewer's spent yeast.

21. The process according to claim 1, wherein the hydrolyzing step is carried out at a temperature of 45-55° C. for 24 and 48 hours.
